(19) United States
(12) Patent Application Publication
(10) Pub. No.: US 2012/0078289 A1
(43) Pub. Date: Mar. 29, 2012

(54) REPOSABLE ENDOSCOPIC FORCEPS SYSTEM

(76) Inventor: STEVE LIVNEH, Amherstburg (CA)

(21) Appl. No.: 13/247,768

(22) Filed: Sep. 28, 2011

Related U.S. Application Data
(60) Provisional application No. 61/387,081, filed on Sep. 28, 2010.

(51) Int. Cl. A61B 17/29

(52) U.S. Cl. 606/205

(57) ABSTRACT

Reposable endoscopic forceps systems are described. The forceps systems may be provided with one or more reposable elements that are intended to be interoperable therewith in various different combinations. By way of a non-limiting example, the reposable elements of the forceps system may be, without limitation: (1) a threaded reposable tip insert wherein the associated tube and adaptor are retained as reusable elements and may thus be permanently attached to a handle; (2) a cartridge system, including a jaw assembly operably associated therewith, a retaining tube, an activation shaft, and an adaptor; and (3) a threaded tip insert and a detachable tube, thus allowing thorough cleaning of both the insert and the tube.

### CROSS-REFERENCE TO RELATED APPLICATION

The instant application claims priority to U.S. Provisional Patent Application Ser. No. 61/387,081, filed Sep. 28, 2010, the entire specification of which is expressly incorporated herein by reference.

### FIELD OF INVENTION

The present invention relates generally to forceps systems, and more specifically to reposable endoscopic forceps systems (i.e., including active components of limited use), including those suitable for the field of endoscopic monopolar applications and inert forceps (i.e., hand held endoscopic surgical instruments).

### BACKGROUND OF THE INVENTION

Endoscopy and endoscopic instruments have been a growing trend in the past 30 years or so. The hand instruments, commonly referred to as forceps, are essential for the purpose of performing endoscopic surgery, as they allow the surgeon to operate remote functional jaws within the endoscopic site. As such, the induced cost and efficacy of forceps are of major importance to surgical centers, the surgeons, and the health industry.

Many variants of reusable, single use and reposable forceps have been introduced through the years. The main goals of the designers are safety, efficacy, reduced cost and ease of use, i.e., ergonomics. Unfortunately, these conventional forceps systems have not been entirely satisfactory in that they are unable to achieve all, or most, of the aforementioned goals.

Accordingly, there exists a need for new and improved forceps systems that overcome at least one of the aforementioned shortcomings.

### SUMMARY OF THE INVENTION

In accordance with the general teachings of the present invention, new and improved forceps systems are provided. More specifically, the forceps systems of the present invention may be provided with one or more reposable elements that are intended to be interoperable therewith in various different combinations.

By way of a non-limiting example, the reposable elements of the forceps system of the present invention may be, without limitation: (1) a threaded reposable tip insert wherein the associated tube and adaptor are retained as reusable elements and may thus be permanently attached to a handle; (2) a cartridge system, including a jaw assembly operably associated therewith, a retaining tube, an activation shaft, and an adaptor; and (3) a threaded tip insert and a detachable tube, thus allowing thorough cleaning of both the insert and the tube.

By way of another non-limiting example, a line of products in accordance with the present invention may include, without limitation, the following combinations: (1) a reusable handle with a permanently attached tube, with a reposable threaded tip insert (“RH-RI”); (2) a reusable handle with a reposable cartridge (“RH-RC”) (it should be noted that this handle may accept a reusable tube); and (3) a reusable handle with a reusable, detachable tube (“RH-RT”) (it should be noted that this tube may accept tip inserts and the handle may accept cartridges as well).

In accordance with one embodiment of the present invention, a surgical instrument is provided, comprising: a forceps system having a distal portion and a proximal portion, wherein a forceps assembly is operably associated with the distal portion and an engagement member is operably associated with the proximal portion; a tube system having an area defining a bore formed therein, wherein the bore is selectively operable to receive the proximal portion of the forceps system therein; a handle system, wherein the handle system includes an area defining a receiving slot formed in a surface thereof; and a biasable member operably associated with the handle system, wherein the biasable member is selectively operable to maintain the receiving slot in a first position operable to receive the engagement member; wherein when the proximal portion is advanced into the bore, the biasable member is selectively operable to maintain the receiving slot in a second position so as to secure the engagement member in the receiving slot.

In accordance with an aspect of this embodiment, the forceps assembly includes a pair of jaw members selectively operable to assume an open or a closed position.

In accordance with an aspect of this embodiment, the engagement member includes a ball-shaped member.

In accordance with an aspect of this embodiment, the handle system includes a pair of handle members, wherein one of then handle members is selectively operable to pivot about the other handle member.

In accordance with an aspect of this embodiment, when the ball-shaped member is secured in the receiving slot, one of the handle members pivots about the other handle member so as to maintain the handle members in the open position.

In accordance with an aspect of this embodiment, the forceps system is comprised of a reposable material.

In accordance with an aspect of this embodiment, the tube system is comprised of a reposable material.

In accordance with an aspect of this embodiment, a bushing member is operably associated with the forceps system, wherein the receiving slot is formed on a surface of the bushing member.

In accordance with an aspect of this embodiment, the distal portion of the forceps system is selectively operable to threadingly engage a distal portion of the tube system.

In accordance with a first alternative embodiment of the present invention, a surgical instrument is provided, comprising: a forceps system having a distal portion and a proximal portion, wherein a forceps assembly is operably associated with the distal portion and an engagement member is operably associated with the proximal portion; wherein the forceps assembly includes a pair of jaw members selectively operable to assume an open or a closed position; wherein the engagement member includes a ball-shaped member; a tube system having an area defining a bore formed therein, wherein the bore is selectively operable to receive the proximal portion of the forceps system therein; a handle system, wherein the handle system includes a pair of handle members, wherein one of then handle members is selectively operable to pivot about the other handle member, wherein the handle system includes an area defining a receiving slot formed on a surface thereof; and a biasable member operably associated with the handle system, wherein the biasable member is selectively operable to maintain the receiving slot in a first position operable to receive the ball-shaped member; wherein when the proximal portion is advanced into the bore, the biasable member is selectively operable to maintain the receiving slot in a second position so as to secure the ball-shaped member in the receiving slot; wherein when the ball-shaped member is secured in the receiving slot, one of the handle members pivots about the other handle member so as to maintain the handle members in the open position.

In accordance with an aspect of this embodiment, the forceps system is comprised of a reposable material.

In accordance with an aspect of this embodiment, the tube system is comprised of a reposable material.

In accordance with an aspect of this embodiment, a bushing member is operably associated with the forceps system, wherein the receiving slot is formed on a surface of the bushing member.

In accordance with an aspect of this embodiment, the distal portion of the forceps system is selectively operable to threadingly engage a distal portion of the tube system.

In accordance with a second alternative embodiment of the present invention, a surgical instrument is provided, comprising: a forceps system having a distal portion and a proximal portion, wherein a forceps assembly is operably associated with the distal portion and an engagement member is operably associated with the proximal portion, wherein an area defining a groove is formed on a surface of the proximal portion; a tube system having an area defining a bore formed therein, wherein the bore is selectively operable to receive the proximal portion of the forceps system therein; a handle system, wherein the handle system includes an area defining a receiving slot formed in a surface thereof; a first biasable member operably associated with the handle system, wherein the first biasable member is selectively operable to maintain the receiving slot in a first position operable to receive the engagement member; wherein when the proximal portion is advanced into the bore, the first biasable member is selectively operable to maintain the receiving slot in a second position so as to secure the engagement member in the receiving slot; and a groove engagement/disengagement system selectively operable to engage the groove so as to further secure the forceps system to the handle system and/or disengage the groove so as to permit disengagement of the forceps system from the handle system.

In accordance with an aspect of this embodiment, the groove engagement/disengagement system includes a pin member selectively operable to engage the groove, a second biasable member selectively operable to raise or lower the pin member, and a handle member selectively operable to contact the pin member.

In accordance with an aspect of this embodiment, the handle member is selectively operable to contact the pin member so as to compress the second biasable member so as to force the pin member to disengage from the groove.

In accordance with an aspect of this embodiment, the handle member includes a tapered member extending therefrom.

In accordance with an aspect of this embodiment, the tapered member is selectively operable to contact the pin member so as to compress the second biasable member so as to force the pin member to disengage from the groove.

In accordance with an aspect of this embodiment, the forceps assembly includes a pair of jaw members selectively operable to assume an open or a closed position.

In accordance with an aspect of this embodiment, the engagement member includes a ball-shaped member.

In accordance with an aspect of this embodiment, the handle system includes a pair of handle members, wherein one of then handle members is selectively operable to pivot about the other handle member.

In accordance with an aspect of this embodiment, when the ball-shaped member is secured in the receiving slot, one of the handle members pivots about the other handle member so as to maintain the handle members in the open position.

In accordance with an aspect of this embodiment, the forceps system is comprised of a reposable material.

In accordance with an aspect of this embodiment, the tube system is comprised of a reposable material.

In accordance with an aspect of this embodiment, a bushing member is operably associated with the forceps system, wherein the receiving slot is formed on a surface of the bushing member.

In accordance with an aspect of this embodiment, the distal portion of the forceps system is selectively operable to threadingly engage a distal portion of the tube system.

Further areas of applicability of the present invention will become apparent from the detailed description provided hereinafter. It should be understood that the detailed description and specific examples, while indicating the preferred embodiment of the invention, are intended for purposed of illustration only and are not intended to limit the scope of the invention.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 illustrates a perspective view of an illustrative forceps system, in accordance with an embodiment of the present invention;

FIG. 2 illustrates a perspective view of the forceps system depicted in FIG. 1 wherein a tip insert system is shown partially disengaged therefrom, in accordance with an embodiment of the present invention;

FIG. 3 illustrates a detailed view of the tip insert system depicted in FIG. 2, in accordance with an embodiment of the present invention;

FIG. 4 illustrates a perspective view of the tip insert system of the forceps system depicted in FIG. 1, in accordance with an embodiment of the present invention;

FIG. 5 illustrates a partial exploded view of the ball-portion of the tip insert system and the bushing member of the forceps system depicted in FIG. 1, in accordance with an embodiment of the present invention;

FIG. 6 illustrates another detailed view of the ball-portion of the tip insert system about to engage a bushing member of the forceps system depicted in FIG. 1, in accordance with an embodiment of the present invention;

FIG. 7 illustrates an isolation view of the ball-portion of the tip insert system engaging a bushing member of the forceps system depicted in FIG. 1, wherein a portion of the bushing member has been removed for purposes of illustration, in accordance with an embodiment of the present invention;

FIG. 8 illustrates a detailed view of the ball-portion of the tip insert system engaging a bushing member of the forceps system depicted in FIG. 1, in accordance with an embodiment of the present invention;

FIG. 9 illustrates a perspective view of the forceps system depicted in FIG. 1 wherein the ball portion of the tip insert system is engaged with the bushing member of the forceps system, in accordance with an embodiment of the present invention;

FIG. 10 illustrates a perspective view of a tube assembly of the forceps system depicted in FIG. 1, in accordance with an embodiment of the present invention;

FIG. 11 illustrates a detailed view of a distal portion of the tube assembly depicted in FIG. 10, in accordance with an embodiment of the present invention;

FIG. 12 illustrates a partial elevational view of the tube assembly/handle assembly interface of the forceps system depicted in FIG. 1, in accordance with an embodiment of the present invention;

FIG. 13 illustrates a sectional view taken along line A-A of FIG. 12, in accordance with an embodiment of the present invention;

FIG. 14 illustrates a detailed view of a proximal portion of the tube assembly depicted in FIG. 10, in accordance with an embodiment of the present invention;

FIG. 15 illustrates another detailed view of a distal portion of the tube assembly depicted in FIG. 10, in accordance with an embodiment of the present invention;

FIG. 16 illustrates still another detailed view of a distal portion of the tube assembly depicted in FIG. 10, in accordance with an embodiment of the present invention;

FIG. 17 illustrates a perspective view of an alternative forceps system, in accordance with an alternative embodiment of the present invention;

FIG. 17A illustrates a section view taken along line B-B of FIG. 17, in accordance with an alternative embodiment of the present invention;

FIG. 18 illustrates a perspective view of a handle assembly of the alternative forceps system depicted in FIG. 17, in accordance with an alternative embodiment of the present invention;

FIG. 19 illustrates a perspective view of a tube assembly of the alternative forceps system depicted in FIG. 17, in accordance with an alternative embodiment of the present invention;

FIG. 20 illustrates a detailed view of an engagement/disengagement system for the tube assembly/handle assembly of the alternative forceps system depicted in FIG. 17, in accordance with an alternative embodiment of the present invention;

FIG. 21 illustrates another detailed view of an engagement/disengagement system for the tube assembly/handle assembly of the alternative forceps system depicted in FIG. 17, in accordance with an alternative embodiment of the present invention;

FIG. 22 illustrates an exploded view of the engagement/disengagement system for the tube assembly/handle assembly of the alternative forceps system depicted in FIG. 17, in accordance with an alternative embodiment of the present invention;

FIG. 23 illustrates a perspective view of the engagement/disengagement system and the handle assembly of the alternative forceps system depicted in FIG. 17, in accordance with an alternative embodiment of the present invention;

FIG. 24 illustrates a sectional view taken along line C-C of FIG. 23, in accordance with an alternative embodiment of the present invention;

FIG. 25 illustrates still another detailed view of an engagement/disengagement system for the tube assembly/handle assembly of the alternative forceps system depicted in FIG. 17, in accordance with an alternative embodiment of the present invention;

FIG. 26 illustrates another perspective view of the alternative forceps system, in accordance with an alternative embodiment of the present invention;

FIG. 27 illustrates still yet another detailed view of an engagement/disengagement system for the tube assembly/handle assembly of the alternative forceps system depicted in FIG. 17, in accordance with an alternative embodiment of the present invention; and

FIG. 28 illustrates still another perspective view of the tube assembly of the alternative forceps system depicted in FIG. 17, in accordance with an alternative embodiment of the present invention.

The same reference numerals refer to the same parts throughout the various Figures.

### DETAILED DESCRIPTION OF THE INVENTION

The following description of the preferred embodiment(s) is merely exemplary in nature and is in no way intended to limit the invention, or uses.

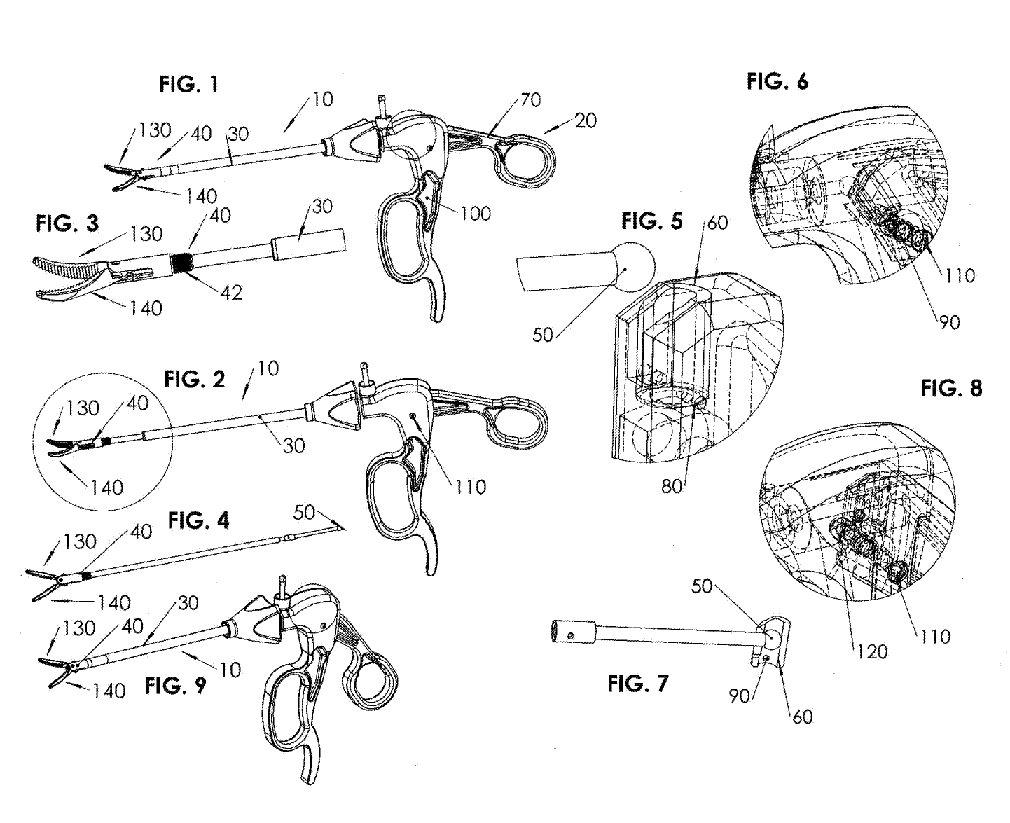

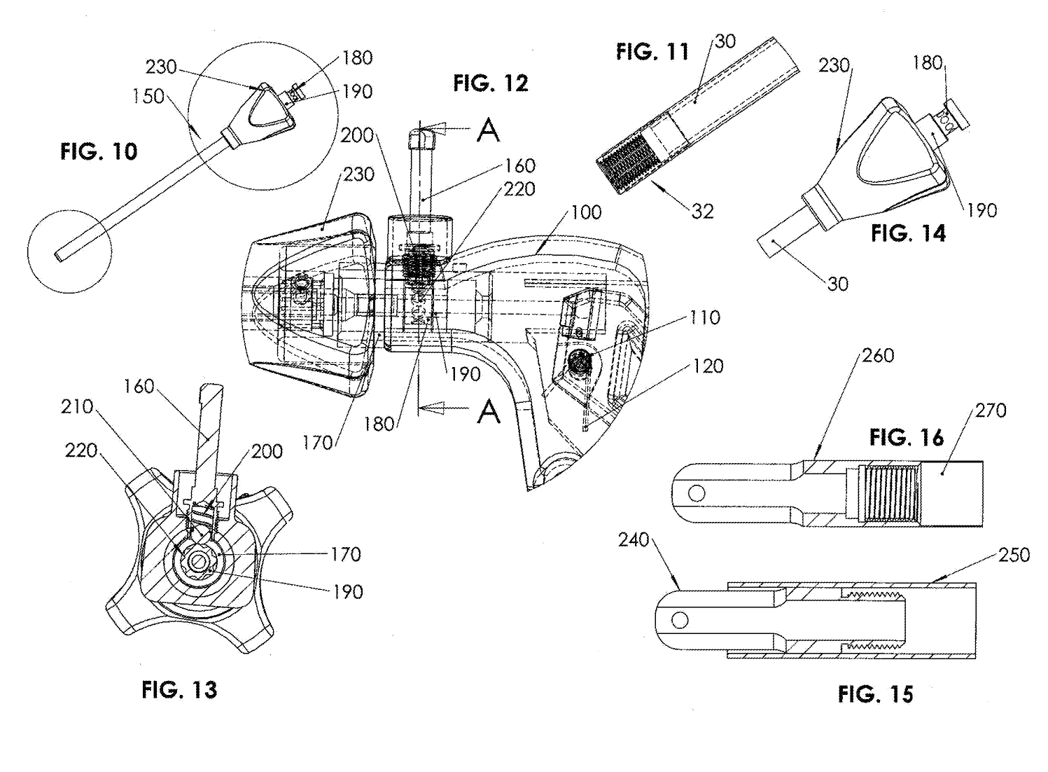

Referring to FIGS. 1-18, there is shown an illustrative assembly of a reposable endoscopic forceps system at 10, in accordance with one embodiment of the present invention. The variant shown herein is the “RH-RI” type, namely a reusable handle system 20, with a permanently attached hollow tube member 30, which is adapted to accept a reposable tip insert system 40.

The tip insert system 40 may be selectively operable to be threaded (e.g., via a threaded portion 42 formed on a surface (e.g., interior and/or exterior thereof) onto, or into, a distal portion of the hollow tube member 30 (e.g., via a threaded portion 32 formed on a surface (e.g., interior and/or exterior) thereof), while simultaneously, a ball-end portion 50 formed on a spaced and opposed opposite end of the tip insert system 40 may engage automatically with a retaining bushing member 60, which may be secured to a proximal handle member 70 by a pin 80 seated in an area defining a hole 90.

The proximal handle member 70 may pivot on a distal handle member 100 via an axle 110 and may be (i.e., with the absence of the ball-end portion 50) fully opened due to the action of a torsion spring member 120, thus positioned to accept the ball-end portion 50 into the retaining bushing member 60. Upon threading of the tip insert system 40 onto or into the hollow tube member 30, which is secured to the distal handle member 100, the ball-end portion 50 may engage the retaining bushing member 60 and may push the proximal handle member 70 forward, thus forcing it to rotate clockwise and hold the handle members 60, 70, respectively, in the fully opened position.

Alternatively, a tube assembly 150 may be secured to the distal handle member 100. For powered forceps systems (e.g., for monopolar applications), a power plug member 160 may be threaded into a sleeve portion 170, positioned within the distal handle member 100 and protruding into an area defining a groove 180 within a tube adaptor 190. A compression spring member 200 and a ball member 210 may be contained within the power plug member 160 and the sleeve portion 170 such that the ball member 210 is partially protruding through the sleeve portion 170 and may be engaged with dimples 220 located at the bottom of the groove 180. A rotatable knob member 230 may be permanently attached to the tube assembly 150. Rotating knob member 230 results in the rotation of the jaw members 130, 140, respectively, and the hollow tube member 30, accompanied by a soft “click” sensation (e.g., an audible and/or tactile indication) when the ball member 210 “softly” locks into a dimple 220, thus helping maintain the orientation of the jaw members 130, 140, respectively, e.g., as selected by the surgeon.

The hollow tube member's 30 distal end may be provided with either a male or a female thread (likewise, the tip insert system 40 may be provided with either a male or female thread). For reposable purposes, a stainless steel tip 240 may be used with an optional polymer jacket 250 that overlaps the threaded portions on the tip insert system 40 and/or the hollow tube member 30 to thus reduce any arcing from the joint. For reposable inserts, a molded tip comprised of insulating material 260, with an integral “skirt” 270, may also be provided.

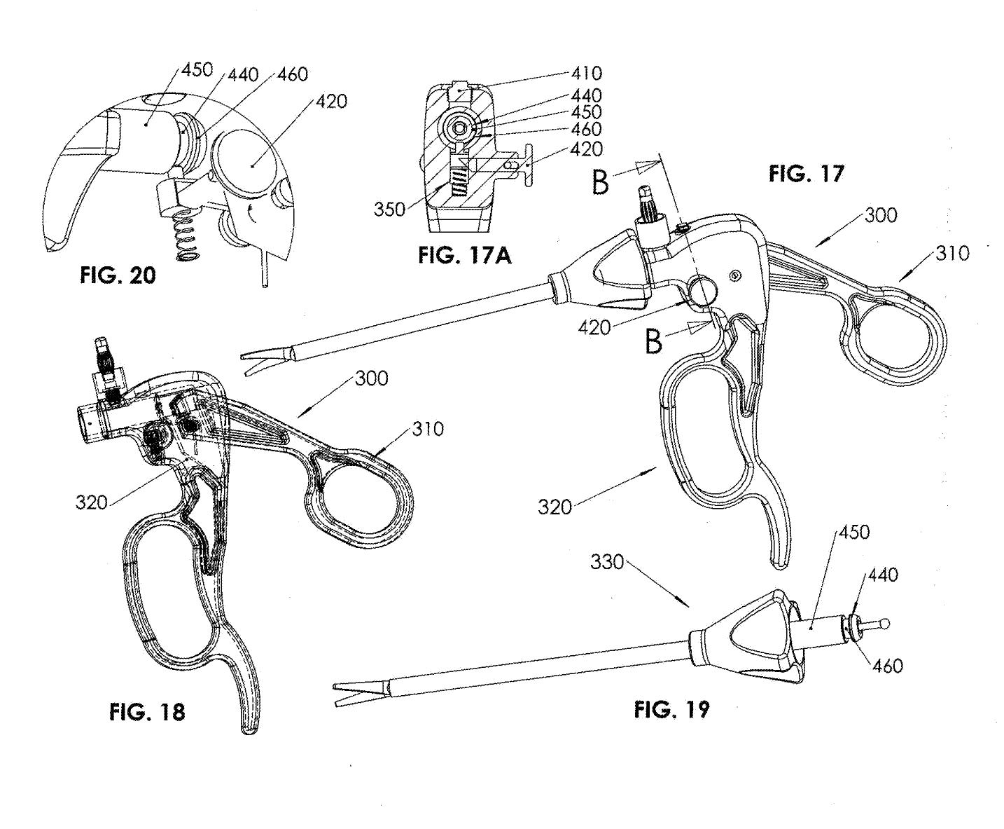

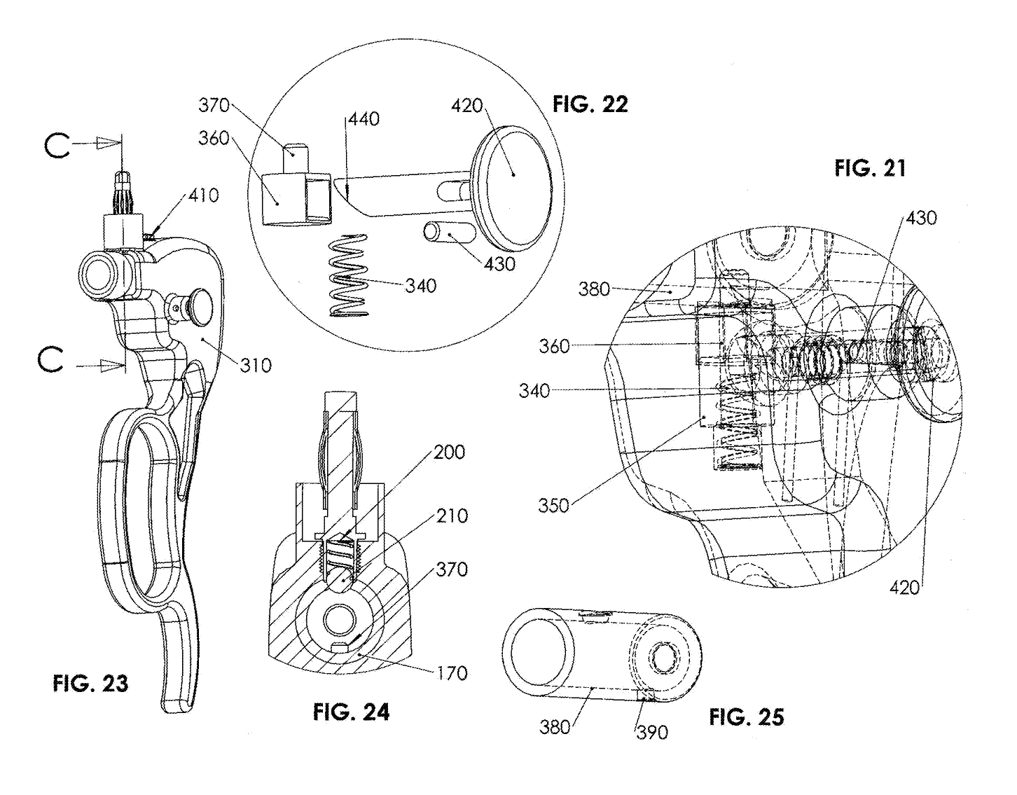

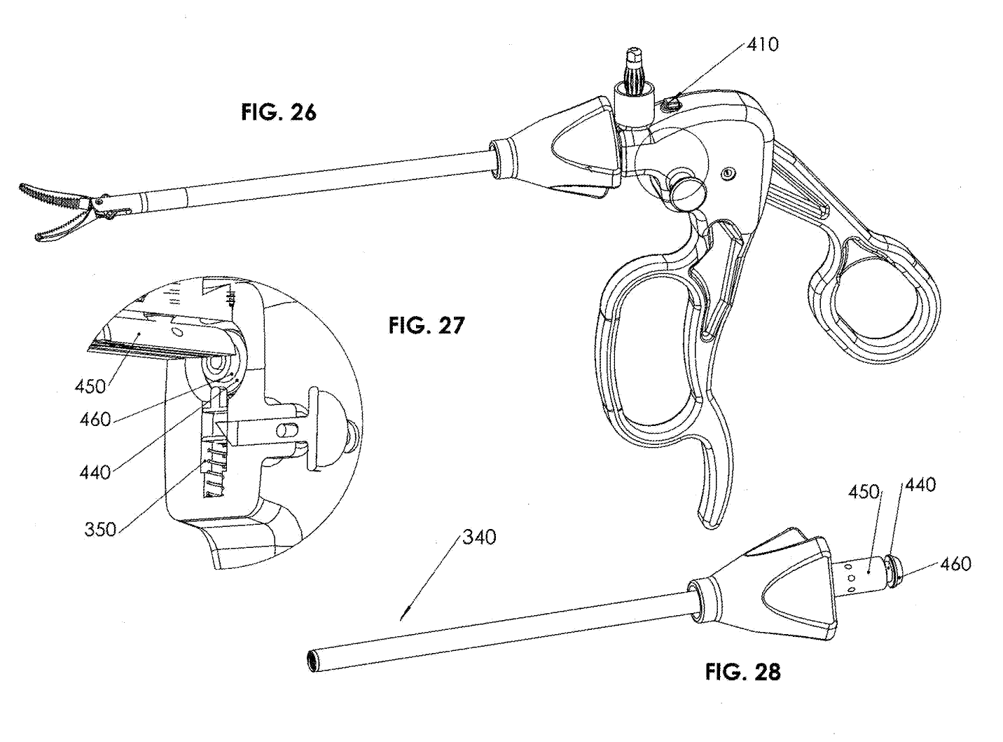

Referring to FIGS. 17-28, there is shown an alternative handle assembly 300, in accordance with an alternative embodiment of the present invention. The proximal handle member 310 is essentially identical to the proximal handle member 70 as depicted in conjunction with the previously described embodiment. However, in this particular embodiment, the distal handle member 320 is different, i.e., it includes an attachment/detachment system that permits the attachment of, e.g., a cartridge assembly 330 and/or a reusable tube assembly 340. The cartridge assembly 330 is essentially a “stand alone” assembly of the previously depicted tube assembly 150 and the tip insert system 40 which are permanently held together and may be attached or detached from the handle member 320 via the system to be described below.

A compression spring member 340 may be partially compressed and seated in a stepped hole 350 in the distal handle member 320, as shown. The spring member 340 exerts upward force, thus pushing a pin assembly 360 with a pin member 370 upwards into a sleeve 380 via an area defining a hole 390. The stepped hole 350 may be formed in the distal handle member 320 by a sliding core-pin. The hole 350 may be sealed with a threaded plug member 410. An activation button 420 may be assembled to the distal handle member 320 via a retaining pin member 430. Depressing the activation button 420 inwardly forces a tapered end portion 440 against the pin assembly 360 and thus forces the pin assembly 360 downwardly, while compressing the spring 340 even further. This action recedes the pin member 370 downwardly through the hole 390 and thus releases the cartridge or the tube, as the case may be, thus allowing them to be pulled distally and detached completely from the distal handle member 320. When attaching the cartridge assembly 330 or the reusable tube reusable tube assembly 340 into the distal handle member 320, the tapered end portion 440 on the adaptor 450 may push the pin member 370 downwardly through the hole 390. The pin member 370 may bounce back into a groove 460 and retain the cartridge assembly 330 or the reusable tube reusable tube assembly 340 until detachment is performed by depressing activation button 420 as previously described above.

Accordingly, the forceps systems of the present invention allows for the efficient and cost effective usage of sharps, such as but not limited to endoscopic scissors, and other jaw types by retaining high cost elements such as the handle assembly, and even the tube assembly, as long-term reusable components. Summarizing, some of the notable features of the present invention are, without limitation: (1) the threaded tip insert is integrated with the full extension of the cable and ball end as a reposable and disposable tip insert; (2) the tip with the overlapping skirt may eliminate arcing from the threaded joint; (3) the attachment/detachment mechanism; and (4) the retaining bushing with the torsion spring and the principle of loading the ball into the bushing, wherein the bushing is tilted forward to accept the ball each time.

The present invention provides a high degree of safety in a well-designed insulation system and ease of grip configuration. The forceps system of the present invention is very user friendly, thus requiring a minimal learning curve. The attachment and detachment of inserts and cartridges is as simple as threading a screw. The tube may be cleaned and sterilized easily and effectively.

While the invention has been described with reference to an exemplary embodiment, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A surgical instrument, comprising:
a forceps system having a distal portion and a proximal portion, wherein a forceps assembly is operably associated with the distal portion and an engagement member is operably associated with the proximal portion;
a tube system having an area defining a bore formed therein, wherein the bore is selectively operable to receive the proximal portion of the forceps system therein;
a handle system, wherein the handle system includes an area defining a receiving slot formed in a surface thereof; and
a biasable member operably associated with the handle system, wherein the biasable member is selectively operable to maintain the receiving slot in a first position operable to receive the engagement member;
wherein when the proximal portion is advanced into the bore, the biasable member is selectively operable to maintain the receiving slot in a second position so as to secure the engagement member in the receiving slot.

2. The surgical instrument according to claim 1, wherein the forceps assembly includes a pair of jaw members selectively operable to assume an open or a closed position.

3. The surgical instrument according to claim 2, wherein the engagement member includes a ball-shaped member.

4. The surgical instrument according to claim 3, wherein the handle system includes a pair of handle members, wherein one of then handle members is selectively operable to pivot about the other handle member.

5. The surgical instrument according to claim 4, wherein when the ball-shaped member is secured in the receiving slot, one of the handle members pivots about the other handle member so as to maintain the handle members in the open position.

6. The surgical instrument according to claim 1, wherein the forceps system is comprised of a reposable material.

7. The surgical instrument according to claim 1, wherein the tube system is comprised of a reposable material.

8. The surgical instrument according to claim 1, further comprising a bushing member operably associated with the forceps system, wherein the receiving slot is formed on a surface of the bushing member.

9. The surgical instrument according to claim 1, wherein the distal portion of the forceps system is selectively operable to threadingly engage a distal portion of the tube system.

10. A surgical instrument, comprising:
a forceps system having a distal portion and a proximal portion, wherein a forceps assembly is operably associated with the distal portion and an engagement member is operably associated with the proximal portion;
wherein the forceps assembly includes a pair of jaw members selectively operable to assume an open or a closed position;
wherein the engagement member includes a ball-shaped member;
a tube system having an area defining a bore formed therein, wherein the bore is selectively operable to receive the proximal portion of the forceps system therein;
a handle system, wherein the handle system includes a pair of handle members, wherein one of then handle members is selectively operable to pivot about the other handle member, wherein the handle system includes an area defining a receiving slot formed in a surface thereof; and
a biasable member operably associated with the handle system, wherein the biasable member is selectively operable to maintain the receiving slot in a first position operable to receive the ball-shaped member;
wherein when the proximal portion is advanced into the bore, the biasable member is selectively operable to maintain the receiving slot in a second position so as to secure the ball-shaped member in the receiving slot;
wherein when the ball-shaped member is secured in the receiving slot, one of the handle members pivots about the other handle member so as to maintain the handle members in the open position.

11. The surgical instrument according to claim 10, wherein the forceps system is comprised of a reposable material.

12. The surgical instrument according to claim 10, wherein the tube system is comprised of a reposable material.

13. The surgical instrument according to claim 10, further comprising a bushing member operably associated with the forceps system, wherein the receiving slot is formed on a surface of the bushing member.

14. The surgical instrument according to claim 10, wherein the distal portion of the forceps system is selectively operable to threadingly engage a distal portion of the tube system.

15. A surgical instrument, comprising:
a forceps system having a distal portion and a proximal portion, wherein a forceps assembly is operably associated with the distal portion and an engagement member is operably associated with the proximal portion, wherein an area defining a groove is formed on a surface of the proximal portion;
a tube system having an area defining a bore formed therein, wherein the bore is selectively operable to receive the proximal portion of the forceps system therein;
a handle system, wherein the handle system includes an area defining a receiving slot formed in a surface thereof;
a first biasable member operably associated with the handle system, wherein the first biasable member is selectively operable to maintain the receiving slot in a first position operable to receive the engagement member;
wherein when the proximal portion is advanced into the bore, the first biasable member is selectively operable to maintain the receiving slot in a second position so as to secure the engagement member in the receiving slot; and
a groove engagement/disengagement system selectively operable to engage the groove so as to further secure the forceps system to the handle system and/or disengage the groove so as to permit disengagement of the forceps system from the handle system.

16. The surgical instrument according to claim 15, wherein the groove engagement/disengagement system includes a pin member selectively operable to engage the groove, a second biasable member selectively operable to raise or lower the pin member, and a handle member selectively operable to contact the pin member.

17. The surgical instrument according to claim 16, wherein the handle member is selectively operable to contact the pin member so as to compress the second biasable member so as to force the pin member to disengage from the groove.

18. The surgical instrument according to claim 16, wherein the handle member includes a tapered member extending therefrom.

19. The surgical instrument according to claim 18, wherein the tapered member is selectively operable to contact the pin member so as to compress the second biasable member so as to force the pin member to disengage from the groove.

20. The surgical instrument according to claim 15, wherein the forceps assembly includes a pair of jaw members selectively operable to assume an open or a closed position.

21. The surgical instrument according to claim 20, wherein the engagement member includes a ball-shaped member.

22. The surgical instrument according to claim 21, wherein the handle system includes a pair of handle members, wherein one of then handle members is selectively operable to pivot about the other handle member.

23. The surgical instrument according to claim 22, wherein when the ball-shaped member is secured in the receiving slot, one of the handle members pivots about the other handle member so as to maintain the handle members in the open position.

24. The surgical instrument according to claim 15, wherein the forceps system is comprised of a reposable material.

25. The surgical instrument according to claim 15, wherein the tube system is comprised of a reposable material.

26. The surgical instrument according to claim 15, further comprising a bushing member operably associated with the forceps system, wherein the receiving slot is formed on a surface of the bushing member.

27. The surgical instrument according to claim 15, wherein the distal portion of the forceps system is selectively operable to threadingly engage a distal portion of the tube system.
